SEQUENCE LISTING (1 sequence)

SEQ ID NO: 1 - protein, 585 aa, Homo sapiens
MTPPRLFWVWLLVAGTQGVNDGDMRLADGGATNQGRVEIFYRGQWGTVCDNLWDLTDASVVCRALGFENATQALGRAAFGQGSGPIMLDEVQCTGTEASLADCKSLGWLKSNCRHERDAGVVCTNETRSTHTLDLSRELSEALGQIFDSQRGCDLSISVNVQGEDALGFCGHTVILTANLEAQALWKEPGSNVTMSVDAECVPMVRDLLRYFYSRRIDITLSSVKCFHKLASAYGARQLQGYCASLFAILLPQDPSFQMPLDLYAYAVATGDALLEKLCLQFLAWNFEALTQAEAWPSVPTDLLQLLLPRSDLAVPSELALLKAVDTWSWGERASHEEVEGLVEKIRFPMMLPEELFELQFNLSLYWSHEALFQKKTLQALEFHTVPFQLLARYKGLNLTEDTYKPRIYTSPTWSAFVTDSSWSARKSQLVYQSRRGPLVKYSSDYFQAPSDYRYYPYQSFQTPQHPSFLFQDKRVSWSLVYLPTIQSCWNYGFSCSSDELPVLGLTKSGGSDRTIAYENKALMLCEGLFVADVTDFEGWKAAIPSALDTNSSKSTSSFPCPAGHFNGFRTVIRPFYLTNSSGVD

(19) United States
(12) Patent Application Publication
(10) Pub. No.: US 2018/0251559 A1
(43) Pub. Date: Sep. 6, 2018

(54) Methods for the Modulation of LGALS3BP to Treat Systemic Lupus Erythematosus

(71) Applicants: Julie DEMARTINO, Westfield, NJ (US); Jonathan DERRY, Bainbridge Island, WA (US); Jaromir VLACH, Westford, MA (US); Nuruddeem LEWIS, Andover, MA (US); Melinda NENEST, Lowell, MA (US); Shinji OKITSU, Cambridge, MA (US); Merck Patent GmbH, Darmstadt (DE)

(72) Inventors: Julie DEMARTINO, Westfield, NJ (US); Jonathan DERRY, Bainbridge Island, WA (US); Jaromir VLACH, Westford, MA (US); Nuruddeen LEWIS, Andover, MA (US); Melinda GENEST, Lowell, MA (US); Shinji OKITSU, Cambridge, MA (US)

(21) Appl. No.: 15/755,692

(22) PCT Filed: Aug. 30, 2016
(86) PCT No.: PCT/US16/49378

§ 371 Date: Feb. 27, 2018

Related U.S. Application Data
(60) Provisional application No. 62/212,163, filed on Aug. 31, 2015.

(51) Int. Cl. C07K 16/28; A61P 13/12

(52) U.S. Cl. CPC C07K 16/2851; A61P 13/12; A61P 37/02; C07K 2317/76; C07K 2317/73

(57) ABSTRACT

Embodiments of the present invention describe methods for modulating LGALS3BP and the use of antibodies to the same in the treatment of autoimmune diseases including systemic lupus erythematosus and lupus nephritis.

### PRIORITY CLAIM

The instant PCT patent application claims priority to U.S. provisional patent application Ser. No. 62/212,163 filed on Aug. 31, 2015, wherein, said provisional application is expressly incorporated by reference, herein, in its entirety.

### SEQUENCE LISTING

The instant application contains a Sequence Listing which has been filed electronically in ASCII format and is hereby incorporated by reference in its entirety. Said ASCII copy, created on Aug. 23, 2016, is named P15167WO_SEQ_LISTING.txt and is 5,320 bytes in size.

### FIELD OF THE INVENTION

The invention relates generally to methods for modulating (including, but not limited to, decreasing, reducing, inhibiting, suppressing, limiting or controlling) the activity of LGALS3BP under conditions such that the production of autoantibodies associated with a variety of autoimmune pathologies are reduced or, alternatively augmenting and enhancing natural antibody secretion or vaccine responses to pathogenic infectious agents through supplementation with recombinant LGALS3BP.

### BACKGROUND OF THE INVENTION

Failure of the immune system can manifest either through the inability to defend the host against infectious agents or, conversely, through a mistaken recognition of self as a breach of tolerance thus giving rise to autoimmune pathologies. Autoimmune pathologies are generally caused by a combination of genetic and environmental factors and can be grossly classified into pathologies mediated by T cells or B cells. Autoreactive pathogenic T cells recognize a target cell by binding the T-cell receptor to the appropriate combination of MHC I molecule and autoantigen-derived peptides resulting in a direct killing of target cells via a number different mechanisms. Development of type-1 diabetes and primary biliary cirrhosis are representative examples of pathologies mediated by autoreactive T cells.

The common feature of B cell associated autoimmunity is the presence of autoantibodies that are directed against functional structures of the cell (nucleic acids, nuclear proteins, receptors, ion channels). By binding to their targets, autoantibodies can mediate cytotoxic destruction of cells by complement activation and/or antibody-dependent cell-mediated cytotoxicity (ADCC) or by blocking the target's function. Pathogenic autoantibodies mediate development of a number of diseases including Graves' disease (anti-thyroid-stimulating hormone Abs), myasthenia gravis (anti-acetylcholine receptor Abs), vasculitis and Wegener's granulomatosis (anti-ANCA Abs) neuromyelitis optica (anti-aquaporin-4 Abs), primary sclerosing cholangitis (anti-neutrophil cytoplasmic Ab, anti-SM Ab). Other autoimmune diseases are caused by a pathogenic action of immune complexes of autoantibodies with their target molecules, e.g. SLE, Sjoegren's syndrome and lupus nephritis (anti-DNA, anti-RNA, anti-histone, anti-Ro, anti-La, anti-phospholipid Abs), subset of rheumatoid arthritis (anti-citrullinated protein, anti-RF, anti-CarP Abs).

Therapeutic approaches for treatment of autoimmune diseases have a rather limited efficacy. The traditional treatment regimens rely on action of steroids and various cytotoxic and cytostatic immunosuppressants that should eliminate rapidly proliferating autoreactive immune cells and thus slow down development of autoimmune processes. The most commonly used drugs for treatment of autoimmune diseases, i.e., cortisone/prednisone, methotrexate, mycophenolate mofetil, chloroquine and azathioprine exhibit limited therapeutic efficacy and are accompanied by numerous adverse effects.

More targeted approaches focus on elimination of autoantibody production and hold better therapeutic promise. Belimumab (trade name Benlysta, previously known as LymphoStat-B), a human monoclonal antibody that inhibits B-cell activating factor (BAFF), also known as B-lymphocyte stimulator (BlyS), a cytokine important for B-cell differentiation and survival, is an approved therapy for adult patients with active, autoantibody positive SLE, and which demonstrates only modest efficacy. Several other biologic therapies attempting to eliminate B cells and, by consequence, the associated pathogenic autoantibodies have focused on cell surface receptors and molecules that are present on human B cells. The anti-CD20 targeting antibody rituximab (and similarly additional biologics, for example, ocrelizumab, obinutuzumab and ofatumumab) was designed to recognize antibody-producing B cells and eliminate them via ADCC. Although no anti-CD20 antibodies have been approved for treatment of SLE, they are often prescribed off-label for treatment of SLE and other autoimmune diseases. In addition, biologics targeting additional surface molecules on human B cells, CD19 and CD22 (epratuzumab), are or were undergoing clinical development, albeit thus far with limited or no clinical effect. The common drawback of the B cell targeting strategies is thought to be the absence of their targets on the surface long-lived plasma cells. The CD19−/CD38hi/CD138+ plasma cells reside in bone marrow and are the source of the majority of the long-lived Ab responses. Therapeutics that could block their activity or lead to their elimination to suppress pathogenic autoantibody production are not currently identified.

Systemic lupus erythematosus (SLE) is a representative autoimmune disorder characterized by formation of autoantibody-containing immune complexes (ICs) that trigger inflammation, tissue damage and premature mortality. SLE ICs often contain nucleic acids that are recognized by numerous innate immune receptors that can initiate pathological mechanisms leading to production of cytokines, interferons and ultimately to immune responses leading to organ damage. Due to the great clinical diversity and idiopathic nature of SLE, management of idiopathic SLE depends on its specific manifestations and severity. Therefore, medications suggested to treat SLE generally are not necessarily effective for the treatment of all manifestations of and complications resulting from SLE, e.g., LN. LN usually arises early in the disease course, within 5 years of diagnosis. The pathogenesis of LN is believed to derive from deposition of immune complexes in the kidney glomeruli that initiates an inflammatory response. An estimated 30-50% of patients with SLE develop nephritis that requires medical evaluation and treatment. LN is a progressive disease, running a course of clinical exacerbations and remissions.

While many patients fail to respond or respond only partially to the standard of care medications listed above, the long-term use of high doses of corticosteroids and cytotoxic therapies may have profound side effects such as bone marrow depression, increased infections with opportunistic organisms, irreversible ovarian failure, alopecia and increased risk of malignancy. Infectious complications coincident with active SLE and its treatment with immunosuppressive medications are the most common cause of death in patients with SLE. Therefore, there is a need for alternative therapeutic agents to treat SLE, and in preferred embodiments LN, wherein said therapeutic agents are associated with fewer side effects than current standards of care.

### SUMMARY OF THE INVENTION

The subject of this application, LGALS3BP, is identified as a B-cell associated target whose functional blockade leads to elimination of activated B cells as well as long-lived plasma cells. While it is not intended the claimed methods of the present invention be limited to any specific mechanism, B cell activation and production of antibodies is regulated at many levels. In one instance B cells get activated by various T cell-dependent stimuli (e.g., CD40 ligation) as well as T cell-independent stimuli (various TLR ligands, polysaccharides, etc.). As shown in the Experimental section of the instant application, TLR7 agonists provide examples of a B cell stimulant as a representative case of B cell activating agents that can induce production of antibodies.

Autoantibody production is widely observed clinically, yet only a small percentage of the population who produce autoantibodies will develop SLE. Moreover, the autoantibody repertoire in SLE is restricted and seems to be enriched for antibodies that recognize autoantigens on proteins that are associated with nucleic acids. The majority of SLE patients have documented production of antibodies against DNA, RNP or both. Autoantigens associated with nucleic acids activate autoreactive B cells and allow them to escape peripheral tolerance checkpoints and differentiate into autoantibody-secreting cells.

Following antigen recognition and uptake of nucleic acid-cell debris complexes the nucleic acids are recognized, in part, by endosomal toll-like receptors (e.g., TLR3, TLR7, TLR8 and TLR9). Stimulation of TLRs in B cells leads to their activation and maturation and increased production of antibodies as well as numerous cytokines. The relative contribution of individual TLRs in the development of SLE has been observed in many mouse SLE models. Moreover, the activity of TLR7, an RNA receptor, plays a major role and gene knock out as well as use of TLR7 inhibitors significantly attenuates disease progression. Also, increased TLR7 activity either by overexpression of TLR7 gene or by systemic administration of small molecule TLR7 agonists leads to induction of SLE-like symptoms.

Nucleic acids present in SLE immune complexes can also be recognized by TLRs in dendritic cells. Stimulation of TLR7 in plasmacytoid dendritic cells leads to production of large amounts of type I interferon. Type I IFN is a cytokine that is involved in antiviral defense by activating a set of genes (interferon target genes) that contribute to control of the virus spread and preservation of host integrity. These genes are often seen activated in SLE patients. Type I IFN plays a role in activating B cells and their expansion and differentiation into Ig-producing cells.

In view of the key role TLR7 stimulation plays in the activity of B cells, embodiments of the present invention describe screens which identify proteins that can modulate production of antibodies. These screens identified proteins and pathways useful in the pharmacological modulation of autoantibody production in the treatment of SLE. A library of plasmids coding for secreted proteins for transient production of cell culture supernatants enriched for these proteins was used and, subsequently, the activity of these proteins in a cellular system with primary B cells stimulated with a small molecule TLR7 ligand using IgG production as a readout to score efficacy. This screen identified a number of proteins that either increase or decrease production of IgGs. Embodiment of the present invention describe proteins not previously associated with B cell biology which include, in a preferred embodiment, LGALS3BP.

LGALS3BP (Mac2-BP, p90) is a ubiquitously expressed gene that belongs to the scavenger receptor family, originally identified as a protein secreted by certain types of tumor cells LGALS3BP expression levels are closely correlated with tumor progression. Apart from its direct effect on tumor cell proliferation/survival, LGALS3BP can also upregulate expression of vascular endothelial growth factor and promote angiogenesis. Its levels are augmented during HIV-1 infection and its activity is believed to reduce infectivity of HIV-1 through interference with the maturation and incorporation of envelope proteins into virions. Analysis of liver biopsies of hepatitis C patients suggested a direct role of LGALS3BP in hepatitis C-related fibrosis. In addition, increased levels of plasma LGALS3BP were also observed in SLE patients. LGALS3BP may contribute to increased cardiovascular complications in SLE, as it can facilitate thrombus formation and attachment of thrombi to endothelial cells. Serum levels of LGALS3BP were also found to be increased in patients with Behcet's disease and correlated with disease activity.

A variety of proteins that interact with and mediate the function of LGALS3BP have been described, including galectins, lectins, integrins and others. LGALS3BP contains several protein-protein interaction domains (SRCR, BTB, POZ) that are likely involved in numerous interactions with cellular proteins in a cell-specific manner.

In one embodiment of the present invention methods are described, wherein, LGALS3BP promotes IgG production in primary B cells stimulated with TLR7 ligand under conditions such that LGALS3BP-neutralizing antibodies significantly reduce IgG production from B cells stimulated with TLR7 ligand or via BCR-ligation. Transcriptome analysis of various immune cells in SLE revealed that LGALS3BP mRNA levels are increased relative to healthy donors and correlate with expression levels of interferon regulated genes.

While it is not intended that the claimed embodiments of the present invention be limited to any specific mechanism (in particular any suggestion that TLR7 must exert, exclusively, a stimulatory effect) the effects that LGALS3BP exert in IgG production in B cells and provides validation for the use of LGALS3BP neutralizing antibodies in the treatment of SLE, LN and potentially other autoimmune diseases such as rheumatoid arthritis, juvenile rheumatoid arthritis, psoriatic arthritis, diabetes mellitus, myasthenia gravis, vasculitis, primary sclerosing cholangitis, autoimmune thyroiditis, Sjogren's Syndrome, Wegener's granulomatosis, Graves' disease, Hashimoto's thyroiditis, autoimmune thrombocytopenic purpura, anti-phospholipid syndrome, neuromyelitis optica and primary sclerosing cholangitis.

Outside of autoimmunity however, augmentation of a naturally occurring or vaccine-induced pathogen-directed humoral immune responses may be beneficial and indeed may be necessary to provide protective immunity against bacteria, parasites or viruses in an infectious disease setting. In this regard, for example, strategies to enhance the efficacy of recombinant protein subunit vaccines without sacrificing safety are of great interest, because immune responses, elicited by these (i.e. against malaria) are typically of weaker magnitude and durability relative to more potent live attenuated or recombinant vectors. In such cases, recombinant LGALS3BP supplementation to enhance humoral immunity and anti-pathogen responses will be beneficial in supporting host defense.

In one embodiment the present invention describes a method for modulating LGALS3BP in a subject presenting symptoms of an immune disorder, inflammatory response or autoimmune disease comprising administering an anti-LGALS3BP antibody to said subject under conditions such that at least one symptom of said immune disorder, inflammatory response or disease said is improved.

In one embodiment the present invention describes a method for modulating LGALS3BP in a subject presenting symptoms of the disease states consisting essentially of Graves' disease, myasthenia gravis, vasculitis and Wegener's granulomatosis, neuromyelitis optica, primary sclerosing cholangitis, Sjoegren's syndrome, lupus nephritis and rheumatoid arthritis comprising administering an anti-LGALS3BP antibody to said subject under conditions such that at least one symptom of one of said disease states said is improved.

In a preferred embodiment the present invention describes treating a patient with SLE, comprising administering to the patient a therapeutically effective amount of an anti-LGALS3BP antibody. In one embodiment the anti-LGALS3BP antibody is effective to: (a) inhibit progression of nephritis; (b) stabilize nephritis; or, (c) reverse nephritis, in the patient. In another embodiment, the amount of anti-LGALS3BP antibody is effective to (a) inhibit progression of proteinuria; (b) stabilize proteinuria; or, (c) reverse proteinuria, in the patient.

In one embodiment the present invention describes treating a patient with SLE, comprising administering to the patient a therapeutically effective amount of an anti-LGALS3BP antibody at a dose effective to stabilize or decrease, in the patient, a clinical parameter selected from; (a) the patient's blood concentration of urea, creatinine or protein; (b) the patient's urine concentration of protein or blood cells; (c) the patient's urine specific gravity; (d) the amount of the patient's urine; (e) the patient's clearance rate of inulin, creatinine, urea or p-aminohippuric acid; (f) hypertension in the patient; (g) edema in the patient; and, (h) circulating autoantibody levels in the patient.

In one embodiment the present invention describes administration of recombinant LGALS3BP as an adjuvant to enhance the activity of a virally-directed vaccine by augmenting a protective antibody responses.

### BRIEF DESCRIPTION OF THE DRAWINGS

FIG. 1A shows the data from primary human B cells that were isolated and stimulated with a small molecule TLR7 agonist and cultured for 5 days. A library of conditioned cell culture supernatants with secreted proteins was added and IgG secretion and cell viability (CTG, CellTiter-Glo) measured at the end of culture.

FIG. 1B shows data from different cellular subsets which were isolated by FACS from healthy controls (first data point in each cellular subset) and lupus nephritis patients with increasing levels of type I IFN (data points 2-4). RNA expression was analyzed by RNA-seq. Normalized FPKM expression values are presented on the graph.

FIG. 1C shows purified recombinant LGALS3BP that was added to purified human B cells stimulated with small molecule TLR7 agonist, CpG (ODN2006) or anti-IgM/CD40L/CpG (ODN2006). IgG was measure by AlphaLISA 5 days after stimulation.

FIG. 1D shows human PBMCs that were stimulated with small molecule TLR7 agonist and RNA isolated 5 h later. Gene expression analysis was performed by RNA-seq and expression levels analyzed as normalized FPKM values.

FIG. 2A-1 and FIG. 2A-2 show data from B cells stimulated with small molecule TLR7 agonist in the presence of increasing concentrations of purified recombinant LGALS3BP. B cell activation was measured 16 h later by flow cytometry quantifying CD69 expression.

FIG. 2B presents data from experiments, wherein, an anti-LGALS3BP antibody was tested for specificity in a western blot with recombinant LGALS3BP (recLGALS3BP) and human plasma.

FIG. 2C shows localization of LGALS3BP as detected using anti-LGALS3BP antibody compared to CD19 B cell and DAPI nuclear stain.

FIG. 3A-1 and FIG. 3A-2 show data from isolated primary human B cells that were stimulated with small molecule TLR7 agonist in the presence of potential LGALS3BP inhibitors and controls (left). Anti-LGALS3BP antibody was added to primary human B cells activated with CpG or anti-IgM/CD40L/CpG (right). IgG secretion was measured 5 days later by AlphaLISA.

FIG. 3B-1 shows data from primary human B cells that were activated with small molecule TLR7 agonist in the presence of potential LGALS3BP inhibitors and controls. IgM secretion was measured 5 days later by AlphaLISA.

FIG. 3B-2 shows data from primary human B cells that were activated with small molecule TLR7 agonist in the presence of potential LGALS3BP inhibitors and controls. B cell viability was measured 5 days later by CellTiter-Glo.

FIG. 3B-3 shows data from primary human B cells that were activated with small molecule TLR7 agonist in the presence of potential LGALS3BP inhibitors and controls. IL-6 secretion was measured 2 days after stimulation by AlphaLISA.

FIG. 3C-1 shows data from B cell activation in the presence of potential LGALS3BP inhibitors and controls as measured 16 hours after activation by quantification of CD69 expression by flow cytometry.

FIG. 3C-2 shows data from B cell activation in the presence of potential LGALS3BP inhibitors and controls as measured 16 hours after activation by quantification of CD69 expression shown are percentages of cells that have upregulated CD69.

FIG. 3C-3 shows data from B cell activation in the presence of potential LGALS3BP inhibitors and controls as measured 16 hours after activation by quantification of CD69 expression shown are mean fluorescence intensity (MFI) of CD69 detection on all B cells.

FIG. 3D-1 and FIG. 3D-2 show data from experiments, wherein, an anti-LGALS3BP antibody was added to unstimulated primary human B cells and the subsequent viability of these B cells was measured 2 days later using CellTtiter-Glo.

FIG. 4A shows data from experiments, wherein, kidneys and spleens were collected from female MRL/lpr mice at 14 weeks of age (early disease). Tissue homogenates were analyzed by NanoString for expression of LGALS3BP and compared to C57BL/6 healthy control mice. Alternatively, RNA was isolated from blood or spleen samples of mice treated with pristane or PBS or from blood, spleen, or kidney of BXSB-Yaa old diseased mice or young control mice. Presented LGALS3BP gene expression levels were measured by QPCR and normalized to Hprt.

FIG. 4B shows data from experiments, wherein, SJL mice were immunized with proteolipid protein (PLP) to induce experimental autoimmune encephalomyelitis (“EAE”). On day 7 and 14 SJL-PLP EAE diseased mice were euthanized and lumbar spinal cords were collected. RNA was purified and analyzed by NanoString for expression of LGALS3BP and compared to naïve non-immunized healthy control mice. In the experiments described in FIGS. 4A and 4B each experimental group contained 5 mice or more and diseased mice were compared to healthy controls with a non-paired Student's t test. * p<0.05, ** p<0.01, *** p<0.001.

FIG. 4C presents “IFN gene signature scores”. These scores were calculated based on the expression of 5 genes known to be interferon regulated (USP18, IRF7, IFIT1, OAS3, BST2). Mice were then grouped in 4 quartiles based on these scores and plotted against average LGALS3BP expression relative to healthy control mice.

FIG. 5A shows LGALS3BP expression by QPCR using RNA extracted from in vitro differentiated primary human macrophages activated with indicated stimuli for 6 h. Expression between samples was normalized using HPRT1 as a housekeeping gene.

FIG. 5B shows LGALS3BP measured by ELISA in supernatants of in vitro differentiated primary human macrophages activated with indicated stimuli for 20 h.

FIG. 6A shows primary B cells isolated from healthy controls (HC) and SLE patient blood were stimulated with TLR7 agonist in the presence (stim+Ab) or absence (stim only) of anti-LGALS3BP antibody. IgM was measured in cultures after 5 days of stimulation. * P<0.05; ** P<0.01 two-tailed paired student's t test.

FIG. 6B shows primary B cells isolated from healthy controls (HC) and SLE patient blood were stimulated with TLR7 agonist in the presence (stim+Ab) or absence (stim only) of anti-LGALS3BP antibody. IgG was measured in cultures after 5 days of stimulation. * P<0.05; ** P<0.01 two-tailed paired student's t test.

FIG. 7A-1 and FIG. 7A-2 shows data which validates the ability of anti-LGALS3BP antibody treatment to reduce antibody titers irrespective of specificity. B cells from healthy controls (HC) and SLE patients were stimulated with TLR7 agonist for 5 days and cell culture supernatants analyzed for 128 autoantibody specificities (IgM and IgG). Number of autoantigens recognized was calculated as specificities with a signal to noise ratio >3. Specificities with positive signal in unstimulated B cells+anti-LGALS3BP antibody were filtered out.

FIG. 7B shows a heatmap of antibody titers represented as z scores (sample−avgₐₗₗ)/stdₐₗₗ. Each column represents one donor stimulated with TLR7 agonist with (+ Ab) or without (−) anti-LGALS3BP antibody. * P<0.05 two-tailed paired student's t test.

FIG. 8A-1, FIG. 8A-2 and FIG. 8A-3 present data showing that anti-LGALS3BP antibody treatment reduces the viability of plasma cells. Freshly isolated B cells from healthy volunteers were differentiated into plasma cells in a two-step, 7 day protocol in the presence of cytokines driving B cell activation (step 1) and B cell differentiation (step 2). Flow cytometry of in vitro differentiated human antibody secreting cells (ASC), plasmablasts (PB) plasma cells (PC). Cells were pre-gated on CD19⁺ B cells.

FIG. 8B shows day 7 differentiated plasma cells which were cultured in the presence or absence of anti-LGALS3BP antibody. Viability was measured by CellTiter-Glo (ATP production) after 4 days. * P<0.05 two-tailed paired student's t test.

FIG. 9A-1 and FIG. 9A-2 show how anti-LGALS3BP antibody treatment induces apoptosis preferentially in B cells. Freshly isolated PBMCs from healthy donors were incubated in the presence or absence of anti-LGALS3BP antibody (aLGALS3BP), isotype control (Rabbit IgG), glycerol control or hydroxychloroquine analog (HCQ analog) for 3 days. In FIG. 9A-1, Annexin V and 7-AAD were measured by flow cytometry together with markers for B (CD19) and T (CD3) cells.

FIG. 9B-1 and FIG. 9B-2 show average frequencies of Annexin V-positive apoptotic cells from 4 donors. Relative frequencies of B and T cells in total PBMCs. Frequencies were normalized to no treatment control.

FIG. 10A-1, FIG. 10A-2 and FIG. 10A-3 confirm that anti-LGALS3BP antibody SP-2 does not reduce B cell viability or antibody production. Freshly isolated B cells from healthy volunteers were stimulated with TLR7 agonist in the presence or absence of anti-LGALS3BP antibody SP-2 or PBS control for 5 days

FIG. 10B show how IgM and IgG were measured in cell culture supernatants by AlphaLISA, viability of cells by CellTiter-Glo (CTG).

### DETAILED DESCRIPTION

Embodiments of the present invention are based on the role that LGALS3BP plays in IgG production and the implications of the same for the treatment of SLE and, more particularly, LN. These therapeutic embodiments of the present invention are validated by data showing the following. LGALS3BP is one of the most differentially regulated genes between lupus nephritis patients and healthy controls across multiple cell types. LGALS3BP closely correlates with IFN-inducible genes and is upregulated in human PBMCs after TLR7 stimulation. LGALS3BP enhances IgG secretion in ex-vivo stimulated primary human B cells. LGALS3BP is present on the surface of B cells and all other PBMCs. Blockade of LGALS3BP with antibody or lactose abrogates IgG production. LGALS3BP antibody blockade does not require the inhibitory FcγRIIb on B cells. LGALS3BP blockade specifically reduces viability of cultured primary human B cells with only a small effect on primary monocytes or total PBMCs and that LGALS3BP is upregulated in mouse models of SLE and EAE.

An LGALS3BP polypeptide refers to full length polypeptide sequence, as well as subsequences, fragments or portions, and modified forms and variants of LGALS3BP polypeptide, unless the context indicates otherwise. Such LGALS3BP subsequences, fragments, modified forms and variants have at least a part of, a function or activity of an unmodified or reference LGALS3BP protein. In particular embodiments a modified form or variant retains, at least a part of, a function or activity of an unmodified or reference protein. A “functional polypeptide” or “active polypeptide” refers to a modified polypeptide or a subsequence thereof. For example, a functional or active LGALS3BP polypeptide or a subsequence thereof possesses at least one partial function or activity (e.g., biological activity) characteristic of a native wild type or full length counterpart polypeptide, for example LGALS3BP, as disclosed herein, which function or activity can be identified through an assay. Embodiments of the present invention, therefore, contemplate modified forms and variants of LGALS3BP polypeptide sequences, and subsequences, which modified forms or variants typically retain, at least a part of, one or more functions or activities of an unmodified or reference LGALS3BP polypeptide sequence.

As disclosed herein, particular non-limiting examples of a function or activity of LGALS3BP polypeptide is to modulate aberrant immune response, immune disorder, inflammatory response, or inflammation, or an autoimmune response, disorder or disease. In one embodiment said autoimmune disease is SLE. In a preferred embodiment said autoimmune disease is LN. While it is not intended that the present invention be limited to any specific mechanism additional, non-limiting, examples of a function or activity of LGALS3BP polypeptide is to modulate the expression of IgG.

An exemplary full length human LGALS3BP polypeptide sequence (SEQ ID NO: 1) is as follows:

MTPPRLFWVWLLVAGTQGVNDGDMRLADGGATNQGRVEIFYRGQWGTVCD
NLWDLTDASVVCRALGFENATQALGRAAFGQGSGPIMLDEVQCTGTEASL
ADCKSLGWLKSNCRHERDAGVVCTNETRSTHTLDLSRELSEALGQIFDSQ
RGCDLSISVNVQGEDALGFCGHTVILTANLEAQALWKEPGSNVTMSVDAE
CVPMVRDLLRYFYSRRIDITLSSVKCFHKLASAYGARQLQGYCASLFAIL
LPQDPSFQMPLDLYAYAVATGDALLEKLCLQFLAWNFEALTQAEAWPSVP
TDLLQLLLPRSDLAVPSELALLKAVDTWSWGERASHEEVEGLVEKIRFPM
MLPEELFELQFNLSLYWSHEALFQKKTLQALEFHTVPFQLLARYKGLNLT
EDTYKPRIYTSPTWSAFVTDSSWSARKSQLVYQSRRGPLVKYSSDYFQAP
SDYRYYPYQSFQTPQHPSFLFQDKRVSWSLVYLPTIQSCWNYGFSCSSDE
LPVLGLTKSGGSDRTIAYENKALMLCEGLFVADVTDFEGWKAAIPSALDT
NSSKSTSSFPCPAGHFNGFRTVIRPFYLTNSSGVD

### Definitions

A “polypeptide” refers to two, or more, amino acids linked by an amide or equivalent bond. A polypeptide can also be referred to herein, inter alia, as a protein, peptide, or an amino acid sequence. Polypeptides include at least two, or more, amino acids bound by an amide bond, or equivalent. Polypeptides can form intra or intermolecular disulfide bonds. Polypeptides can also form higher order structures, such as multimers or oligomers, with the same or different polypeptide, or other molecules.

The terms “patient” and “subject” are used in this disclosure to refer to a mammal being treated or in need of treatment for a condition such as SLE or LN. The terms include human patients and volunteers, non-human mammals such as a non-human primates, large animal models and rodents.

“Administering” or “administration of” a drug to a patient refers to direct administration, which may be administration to a patient by a medical professional or may be self-administration, and/or indirect administration, which may be the act of prescribing a drug. For example, a physician or clinic that instructs a patient to self-administer a drug or provides a patient with a prescription for a drug is administering the drug to the patient.

The terms “dose” and “dosage” refer to a specific amount of active or therapeutic agent(s) for administration at one time. A “dosage form” is a physically discrete unit that has been packaged or provided as unitary dosages for subjects being treated. It contains a predetermined quantity of active agent calculated to produce the desired onset, tolerability, and therapeutic effect.

A “therapeutically effective amount” of a drug refers to an amount of a drug that, when administered to a patient to treat a conditions such as SLE and LN, will have a beneficial effect, such as alleviation, amelioration, palliation or elimination of one or more symptoms, signs, or laboratory markers associated with the active or pathological form of the condition.

### EXAMPLES

The following examples are intended for illustration only and should not be construed to limit the scope of the claimed invention.

### Example 1: LGALS3BP Enhances IgG Secretion in B Cells Activated with a TLR7 Agonist

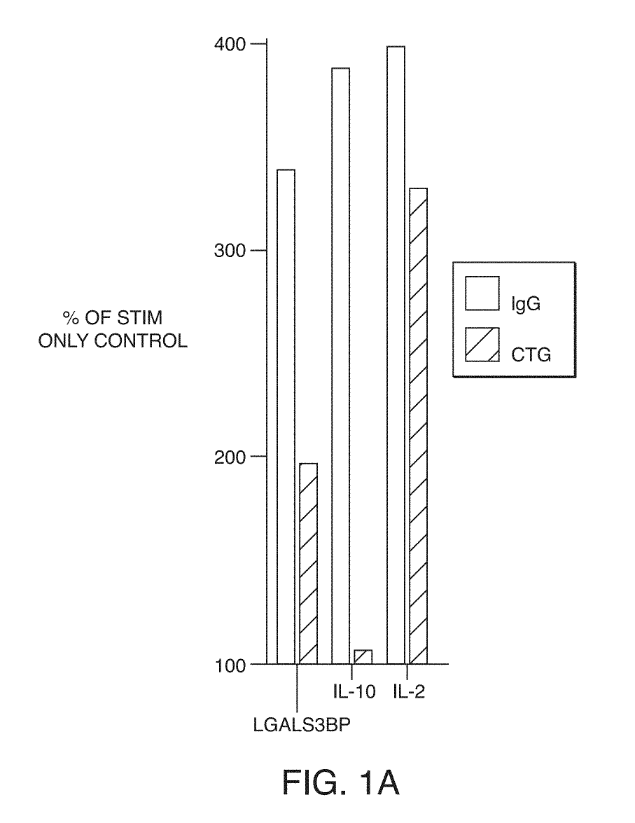

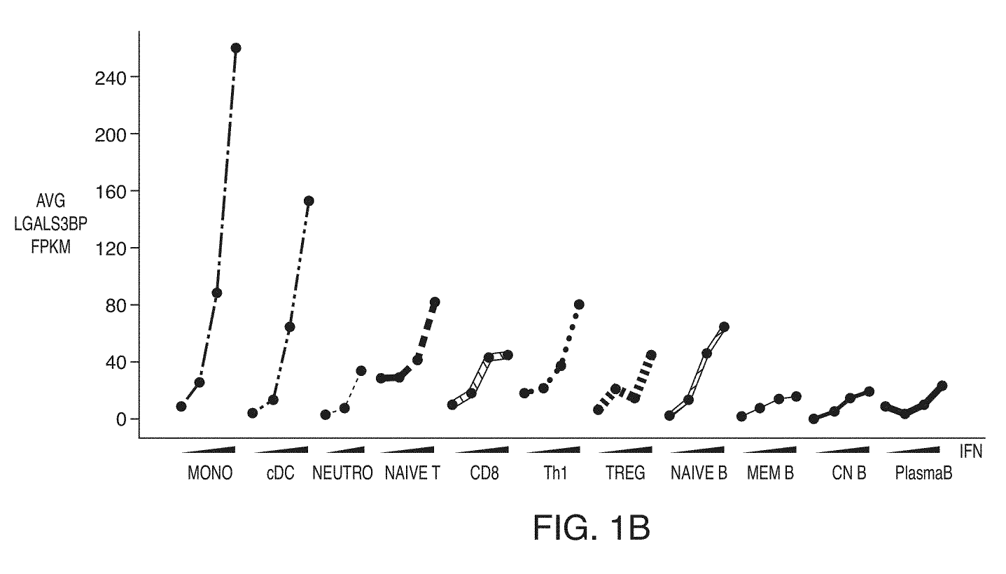

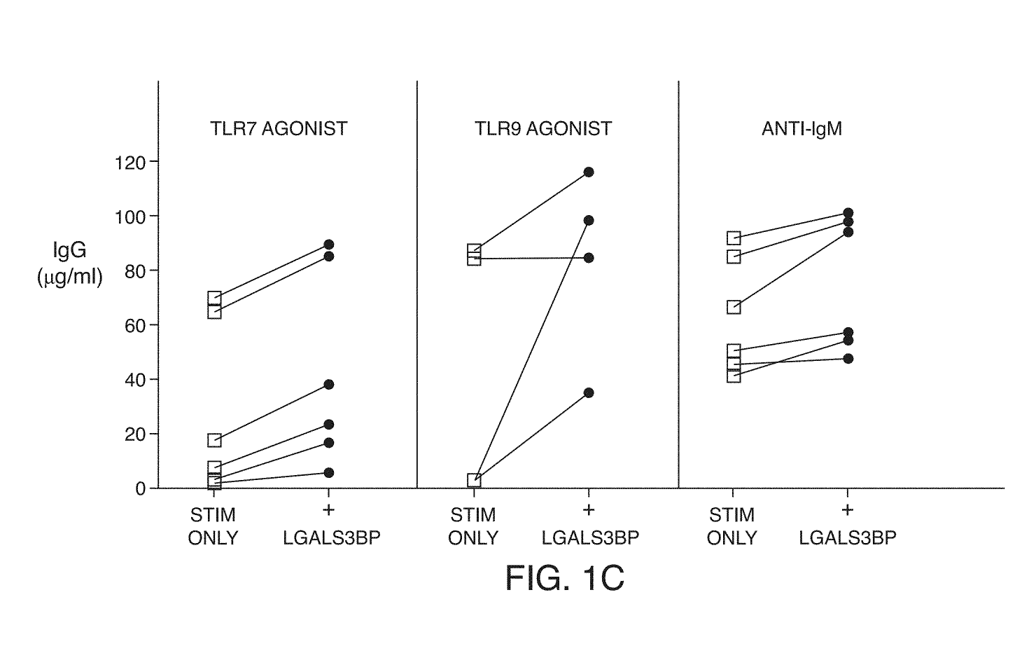

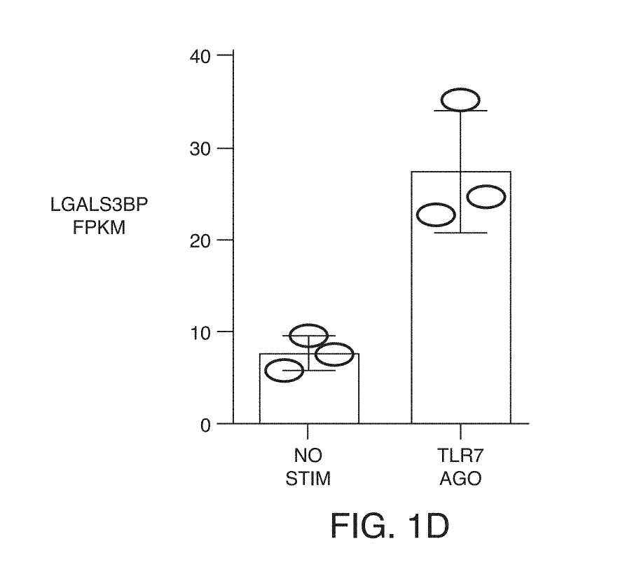

To identify secreted proteins that affect IgG production by B cells a selection of proteins from the human secretome in an IgG secretion assay were screened using primary human B cells. B cells from healthy volunteers were exposed to 1400 recombinantly expressed secreted proteins before activation with a TLR7 small molecule agonist. After 5 days IgG was measured to identify proteins that enhance or inhibit IgG secretion. Besides B cell stimulatory cytokines such as IL-2 and IL10, this experiment demonstrated that LGALS3BP enhanced IgG secretion by 4.1-fold, while cell viability and metabolic activity (ATP measured by CellTiter-Glo assay) doubled (FIG. 1a). LGALS3BP was independently identified as the most differentially regulated gene in blood from lupus nephritis patients compared to healthy volunteers. LGALS3BP was upregulated in all cell types analyzed and correlated with the patient's interferon signature (FIG. 1b).

The enhanced IgG production (1.6-fold) was confirmed using purified recombinant LGALS3BP on B cells from 6 more healthy volunteer human subjects (FIG. 1c). Similar increases in IgG were observed when B cells were stimulated with the TLR9 agonist CpG (1.9-fold) or an activation cocktail with anti-IgM, CD40L and CpG (1.2-fold). PBMCs were simulated from healthy volunteers with a small molecule agonist to test if the activation protocol could enhance LGALS3BP expression in vitro (FIG. 1b). Baseline expression values were comparable to those found in cells directly ex vivo. TLR7 stimulation did increase the expression levels by more than 3-fold. This finding provides an explanation for the variable effect the addition of exogenous LGALS3BP had on B cells from different donors. LGALS3BP was identified as one of the most differentially expressed gene in different immune cell types from LN patients compared to healthy volunteers and found an enhancing role for the secreted protein in antibody production.

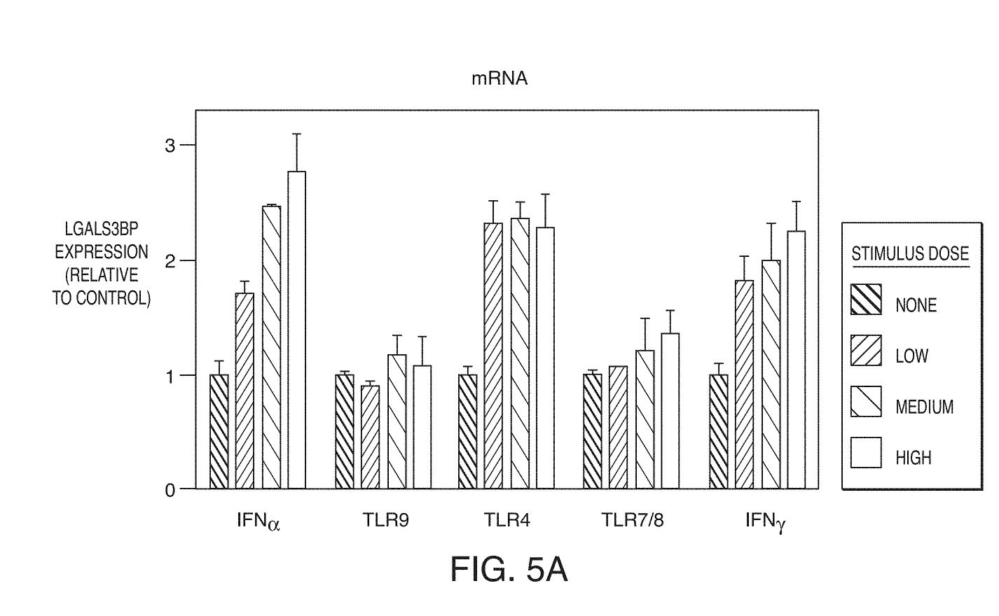

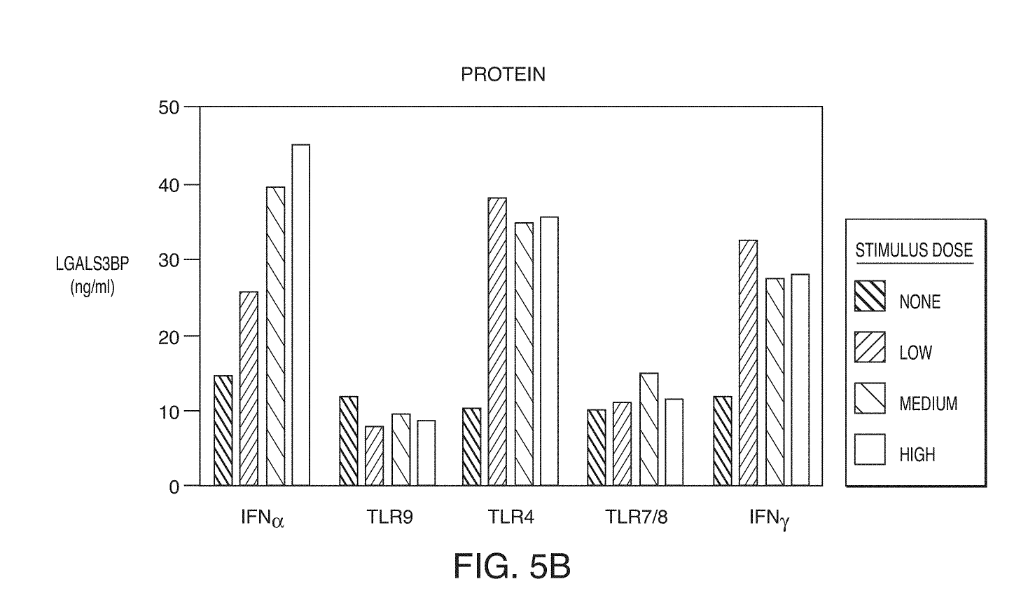

LGALS3BP has an IRF binding site consistent with regulation by type I interferons. To determine which pathways can induce LGALS3BP expression, primary human monocytes were differentiated into macrophages in vitro and subsequently were stimulated with IFN-α, IFN-γ, TLR4 agonist (LPS), TLR7/8 agonist (resiquimod) and TLR9 agonist (CpG). INF-α, IFN-γ and LPS induced LGALS3BP mRNA expression (FIG. 5A) and increased secretion of the protein (FIG. 5B). All stimuli induced secretion of IL-6. This indicates that not only type I interferons can drive LGALS3BP expression but also IFN-γ and other innate triggers.

Based on location of histone acetylation sites, LGALS3BP expression is regulated by factors binding to 4 different regions in the LGALS3BP gene: at the promoter start site, in an upstream enhancer (region 5 K upstream), in an intronic site, or in the 3′ UTR. Motif scanning by 3 different methods identified likely immune-relevant transcriptional regulators. IRFs, AP-1, and STATs as well as other important factors such as NF-KB were found in and around the LGALS3BP gene locus. Prediction of transcription factor binding suggests that LGALS3BP expression is regulated by interferons through interferon regulatory factors (IRFs) as well as other immune stimuli that activate STATs, NF-kB, and AP-1.

### Example 2: LGALS3BP is Present on the B Cell Surface but does not Increase B Cell Activation

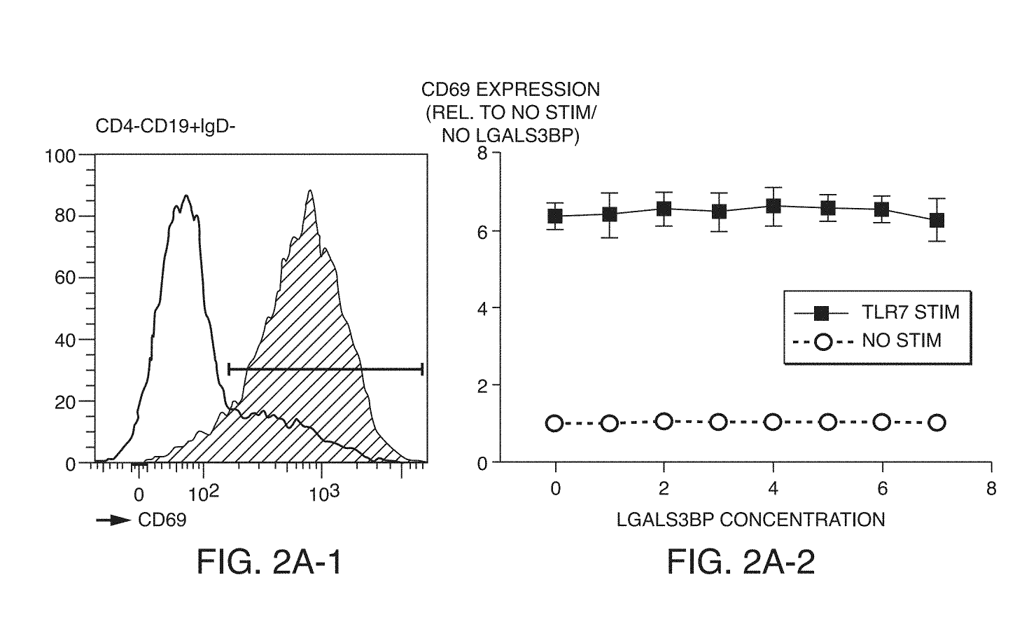

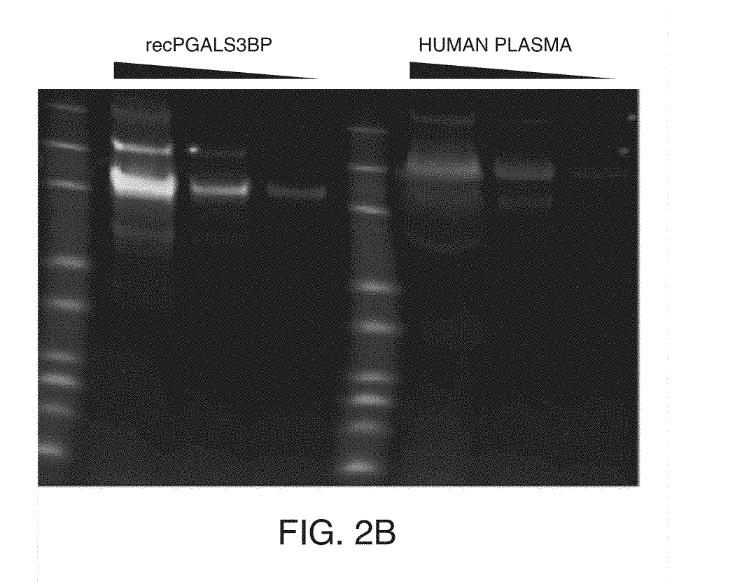

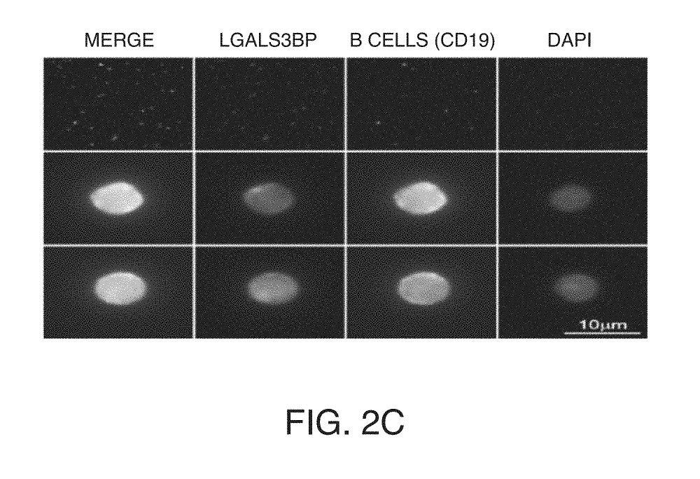

To investigate if addition of LGALS3BP affects activation of naïve B cells CD69 expression was measured 16 h after stimulation with TLR7 agonist. All B cells had increased CD69 expression compared to non-stimulated cells but no change was seen upon addition of various concentrations of recombinant LGALS3BP (FIG. 2A-1 and FIG. 2A-2). The localization of endogenous LGALS3BP in primary human B cells with an antibody specific for LGALS3BP was then evaluated (FIG. 2b). These studies confirmed that LGALS3BP is present on the B cell surface as well as on all other cell types found in PBMCs (FIG. 2c).

### Example 3: Anti-LGALS3BP Inhibits IgG Secretion Through Induction of B Cell and Plasma Cell Apoptosis

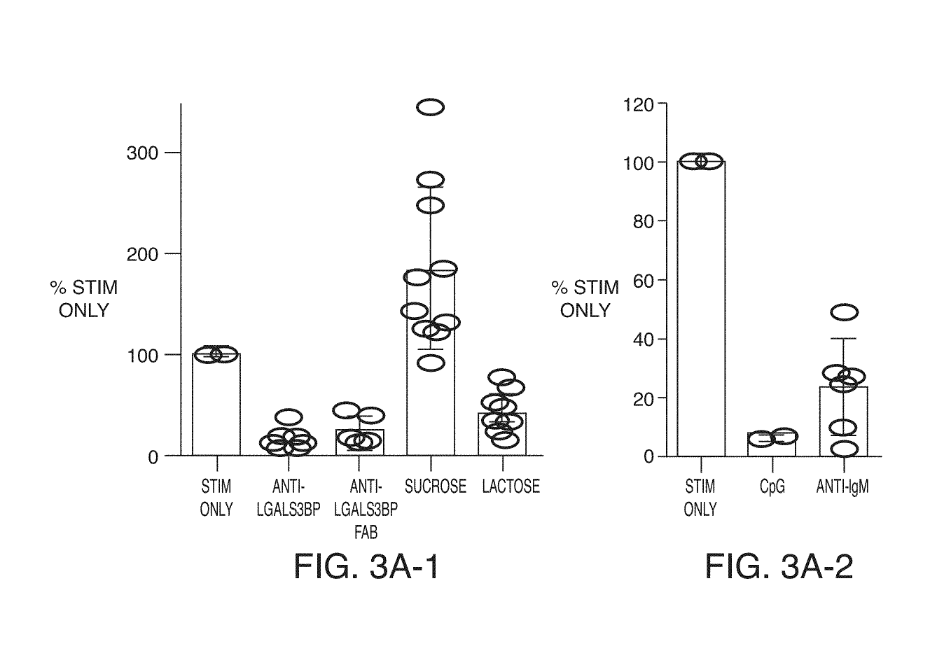

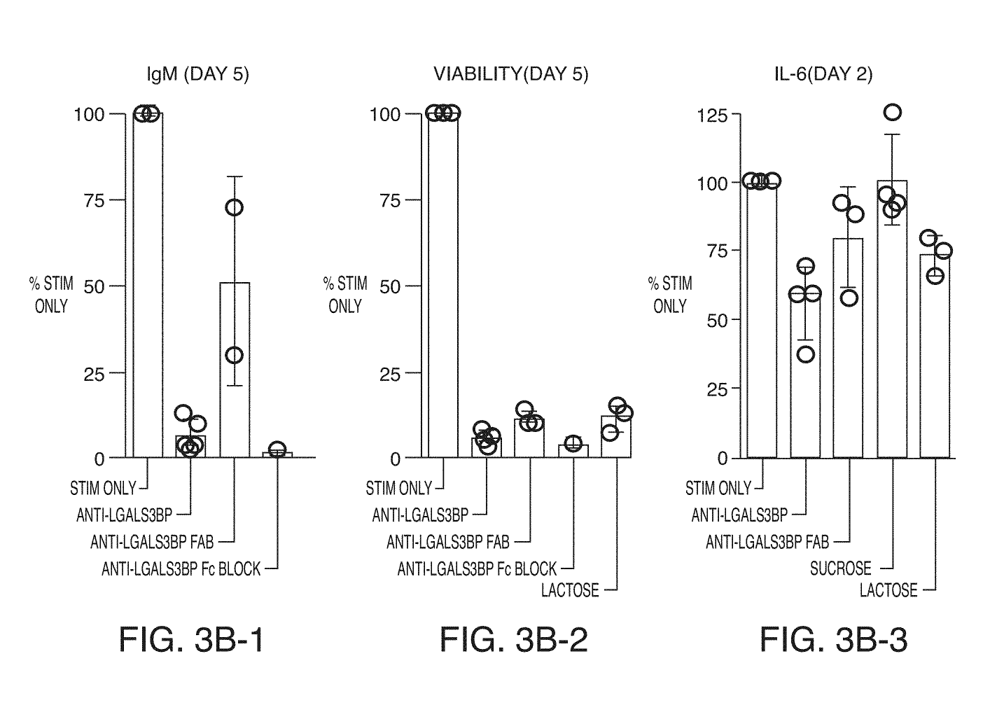

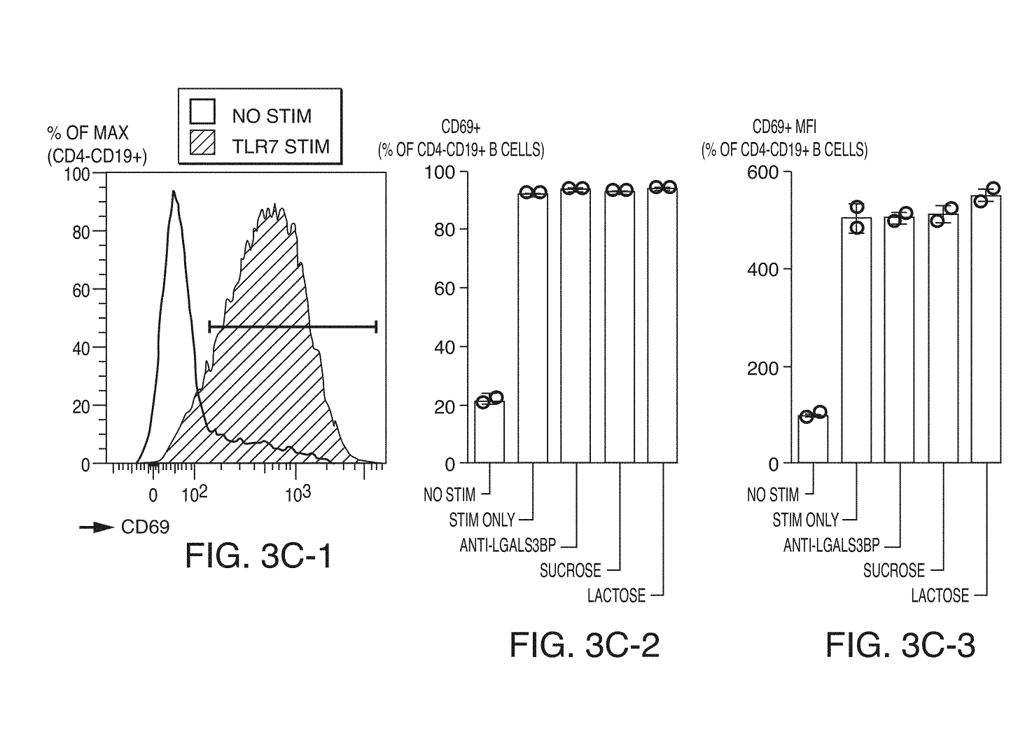

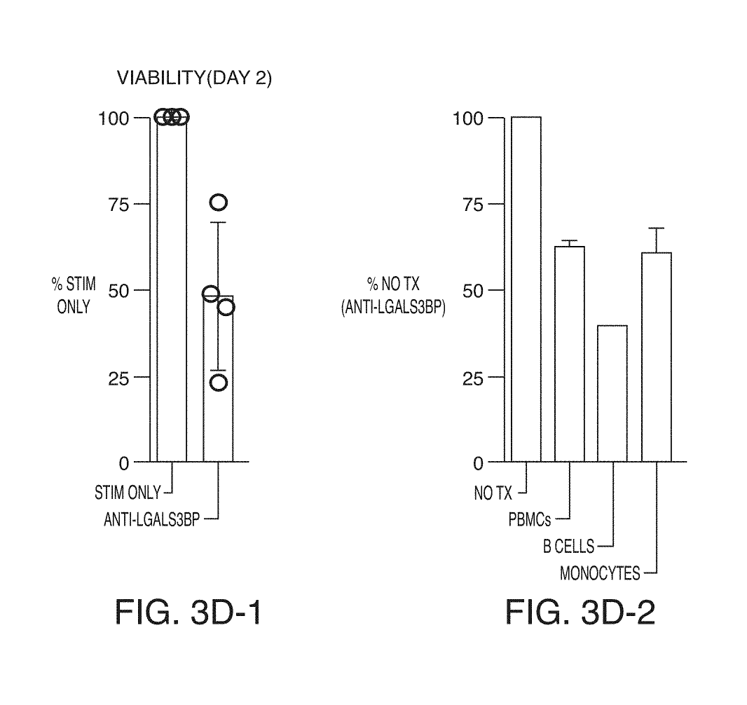

The effect of anti-LGALS3BP antibodies on IgG secretion by primary human B cells was evaluated. IgG secretion by TLR7 activated B cells was inhibited by almost 90% in presence of anti-LGALS3BP antibody or anti-LGALS3BP F(ab′)₂ (74%) to exclude inhibition through FcγRIIb present on B cells (FIGS. 3A-1 and 3A-2). Lactose, a known ligand for LGALS3BP had the same but weaker effect (59% inhibition), while sucrose did not inhibit IgG secretion. The same inhibitory effect of the LGALS3BP antibody was observed when B cells were activated with CpG (94%) or anti-IgM/CD40L/CpG (77%). IgM secretion was inhibited by antibody blockade as well excluding a role of LGALS3BP in isotype switching (FIG. 3B-1, FIG. 3B-2 and FIG. 3B-3). Measuring ATP as a readout for cell number and viability showed a close correlation with IgG secretion, thereby, implicating LGALS3BP in B cell survival and/or proliferation. IL-6 secretion was measured to investigate if LGALS3BP blockade interferes with TLR7 activation and signaling thereby reducing B cell proliferation. A 37% decrease in IL-6 production was observed 48 h after B cell stimulation in the presence of anti-LGALS3BP antibody. This reduction was LGALS3BP specific and not mediated through FcγRIIb given the same effect was measured in the presence of Fc block or with anti-LGALS3BP F(ab′)₂. Lactose also had the same effect, thereby, excluding a direct effect of the antibody through cross-linking the surface-bound protein. Non-stimulated primary human B cells do not proliferate and have limited survival in vitro. To test if anti-LGALS3BP antibodies reduce B cell survival by blocking B cell activation CD69 upregulation was measured 16 h after activation with TLR7 agonist (FIG. 3C-1, FIG. 3C-2 and FIG. 3C-3). No difference in percentage of CD69⁺ activated cells or expression levels of CD69 was observed when an anti-LGALS3BP antibody was added. LGALS3BP blockade inhibits IgG secretion independent of the stimulation protocol used. To determine if LGALS3BP blockade has an effect on B cell survival in the absence of stimulation additional experiments were conducted. Adding the antibody to non-stimulated B cells reduced viability by 66% (FIG. 3D-1 and FIG. 3D-2). This effect was most pronounced in B cells. Anti-LGALS3BP treatment of total PBMCs or monocytes showed a 37.5% and 39% reduction in viability. Together these results confirm an anti-apoptotic role of LGALS3BP during B cell homeostasis, activation, proliferation and differentiation.

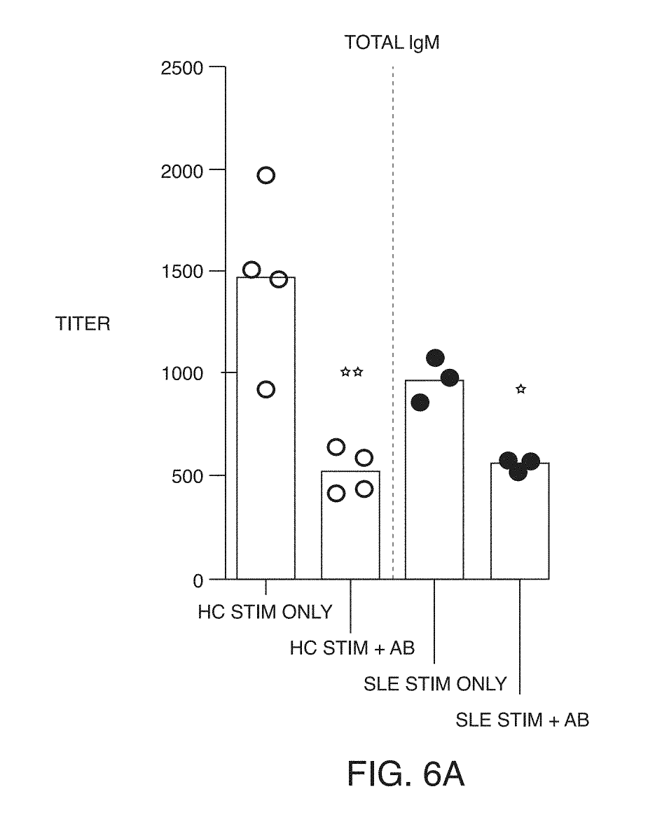

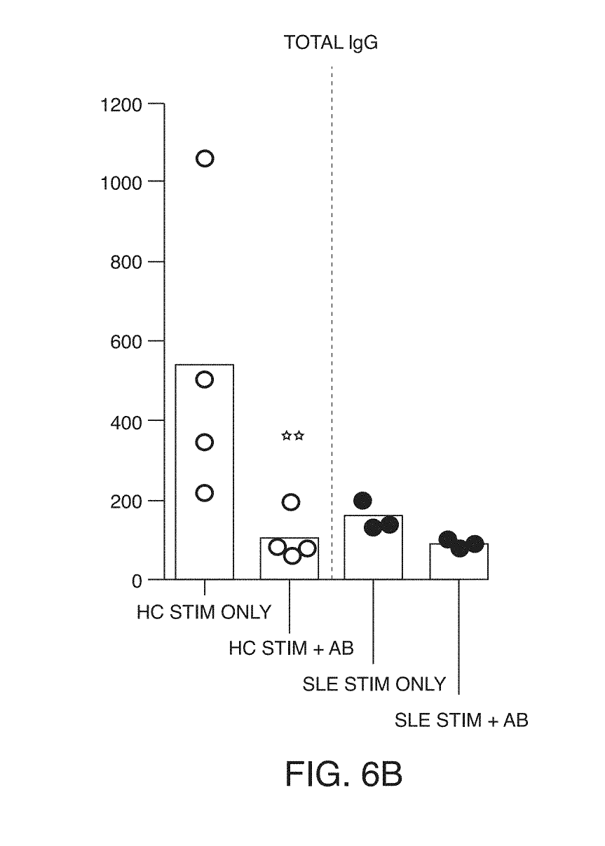

Dysregulated B cell tolerance is a key driver of SLE pathogenesis. To address if anti-LGALS3BP treatment has the same effect on SLE B cells as observed in B cells from healthy donors, the B cell stimulation experiments were repeated in B cells from SLE donors. A significant reduction in IgM production was observed when the cells were stimulated with TLR7 agonist in the presence of anti-LGALS3BP antibody (FIG. 6A and FIG. 6B). There was reduction in IgG secretion, although not significant, accounted for due to the fact that B cells from SLE donors did not raise much IgG in response to TLR7 stimulation. These experiments confirm that the inhibitory effect of anti-LGALS3BP treatment is conserved in SLE B cells.

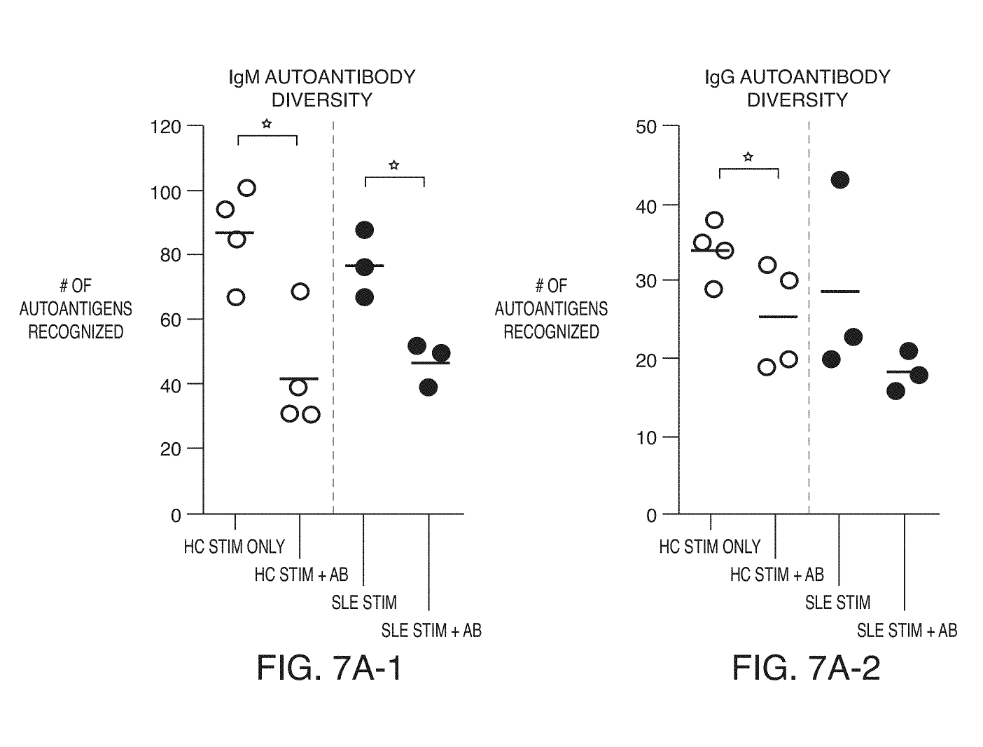

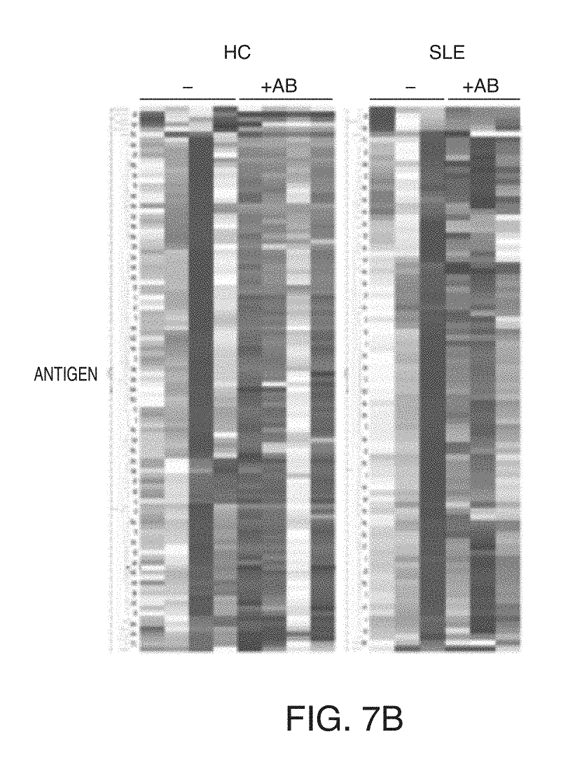

Supernatants from TLR7-stimulated B cells on a 128 autoantigen protein microarray were analyzed (Table 1). Anti-LGALS3BP treatment reduced the number of autoantigens recognized by IgM antibodies (FIG. 7B) and uniformly reduced the IgM titers of all autoantigens, confirming that no specificity escapes anti-LGALS3BP treatment (FIG. 7B). These data confirm that anti-LGALS3BP treatment uniformly reduces antibody production by healthy as well as SLE patient B cells irrespective of specificity.

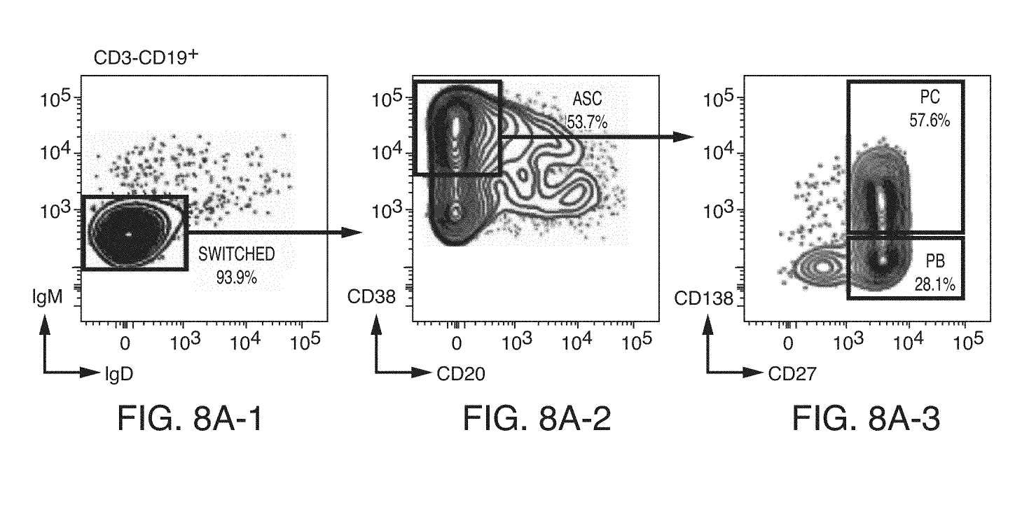

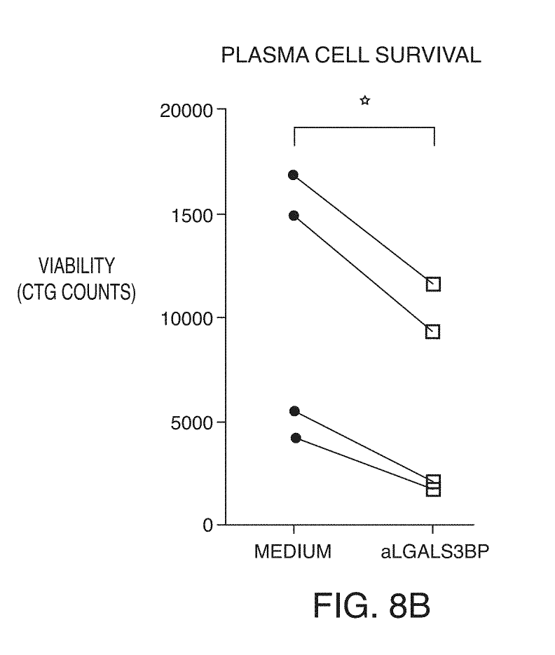

SLE patients usually have pre-existing long-lived plasma cells at the time when diagnosed with the disease. Treatments that deplete B cells are able to reduce antibody titers depending on the specificity. dsDNA-specific antibodies for example are reduced with B cell depletion, while others, such as RNP-specific ones remain elevated. Long-lived plasma cells, on the other hand, are not depleted and continue to secrete antibodies. An in vitro system to differentiate plasma cells from primary human B cells from healthy donors was designed to test if anti-LGALS3BP treatment has an effect on plasma cell viability (FIG. 8A-1, FIG. 8A-2 and FIG. 8A-3). The differentiated plasma cells were then exposed to anti-LGALS3BP antibodies for 4 days and viability was assessed indirectly by measuring ATP production. A significant reduction in plasma cell viability was observed, thereby, validating the therapeutic effect of anti-LGALS3BP treatment on long-lived plasma cells (FIG. 8B).

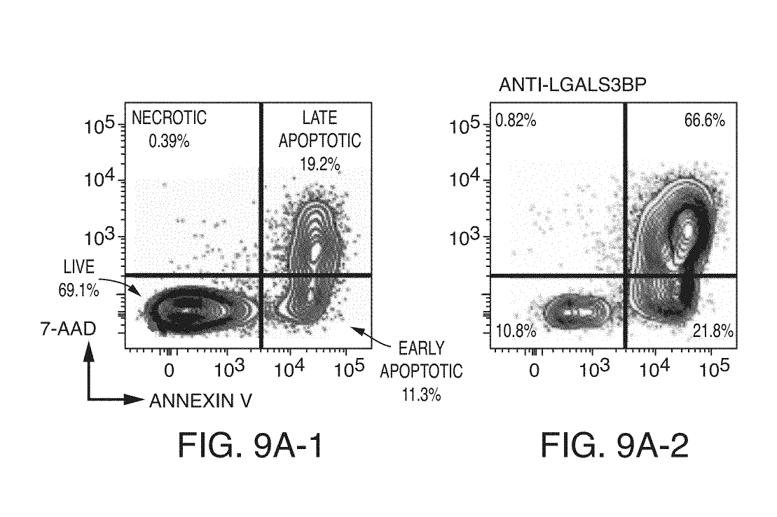

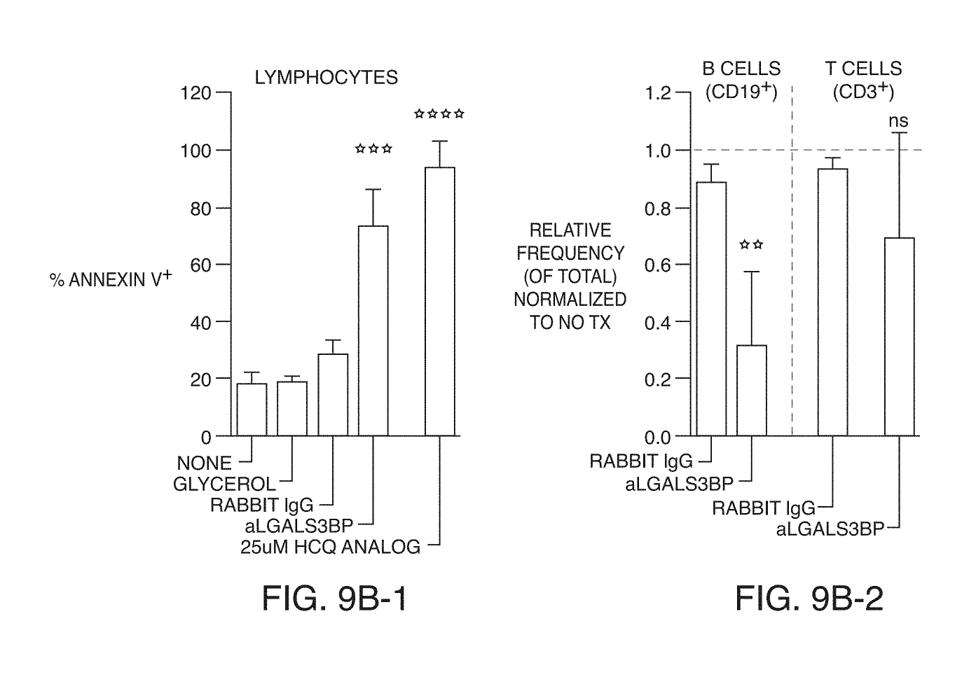

In order to determine if this reduced viability was due to necrosis or apoptosis of the targeted cells, PBMCs from healthy donors were incubated with anti-LGALS3BP antibodies for 4 days and subsequently annexin V surface expression and cell permeability (7-AAD) were measured by flow cytometry. Anti-LGALS3BP treatment induced expression of annexin V, which is consistent with cell death by apoptosis (FIG. 9A-1, FIG. 9A-2, FIG. 9B-1 and FIG. 9B-2). Glycerol or control rabbit IgG did not produce the same effect, while high doses of a hydroxychloroquine analog also induced apoptosis. Comparing the frequency of B and T cells, the treatment affected B cells more than T cells in accordance with the prior observation that PBMCs or monocytes are not as susceptible to treatment as B cells.

These results confirm an anti-apoptotic role of LGALS3BP during B cell homeostasis, activation, proliferation and differentiation.

TABLE 1
List of Antigens on the Autoantigen Array

| | | | |
|---|---|---|---|
| Aggrecan | dsRNA | La/SSB | Ro/SSA (60 KDa) |
| Alpha Fodrin (Sptan1) | dsDNA | Laminin | S100 |
| Alpha-actinin | EBNA1 | LC1 | Scl-70 |
| Amyloid | Elastin | LKM1 | Sm |
| AQP4 recombinant | Entaktin EDTA | M2 antigen | Sm/RNP |
| BP1 | Factor I | Matrigel | SmD |
| C1q | Factor P | MDA5 | SmD1 |
| Cardiolipin | Factor B | Mi-2 | SmD2 |
| CENP-A | Factor D | Mitochondrial antigen | SmD3 |
| CENP-B | Factor H | MPO | SP100 |
| Chondroitin Sulfate C | Fibrinogen IV | Muscarinic receptor | Sphingomyelin |
| Chromatin | Fibrinogen S | Myelin basic protein (MBP) | SPR54 |
| Collagen I | Fibronectin | Myelin-associated glycoprotein-FC | ssDNA |
| Collagen II | GBM (disso) | Myosin | T1F1 GAMMACollagen |
| Collagen III | Genomic DNA | Nucleolin | Thyroglobulin |
| Collagen IV | Gliadin (IgG) | Nucleosome antigen | TNFa |
| Collagen V | Glycated Albumin | Nup62 | Topoisomerase I |
| Collagen VI | GP2 | PCNA | TPO |
| Complement C1q | gP210 | Peroxiredoxin 1 | TTG |
| Complement C3 | Histone H1 | Phophatidylinositol | U1-snRNP-68 |
| Complement C3a | Histone H2A | PL-12 | U1-snRNP-A |
| Complement C3b | Histone H2B | PL-7 | U1-snRNP-BB′ |
| Complement C4 | Histone H3 | PM/Scl-100 | U1-snRNP-C |
| Complement C5 | Histone H4 | PM/Scl-75 | Vimentin |
| Complement C6 | Hemocyanin | POLB | Vitronectin |
| Complement C7 | Heparan HSPG | PR3 | β2-glycoprotein I |
| Complement C8 | Heparin | Proteoglycan | β2-microglobulin |
| Complement C9 | Heparan Sulfate | Prothrombin protein | IgA-human and mouse |
| CPR antigen (human) | Histone (total) | Ribo phosphoprotein P1 | IgE-human |
| Cytochrome C | Intrinsic Factor | Ribo phosphoprotein P2 | IgG-human and mouse |
| Decorin-bovine | Jo-1 | Ribo phosphoprotein P0 | IgM-human and mouse |
| DGPS | KU (P70/P80) | Ro/SSA (52 KDa) | Anti-IgG, IgA and anti-IgM |

### Example 4: LGALS3BP Expression is Increased in Mouse Models of SLE and EAE Model

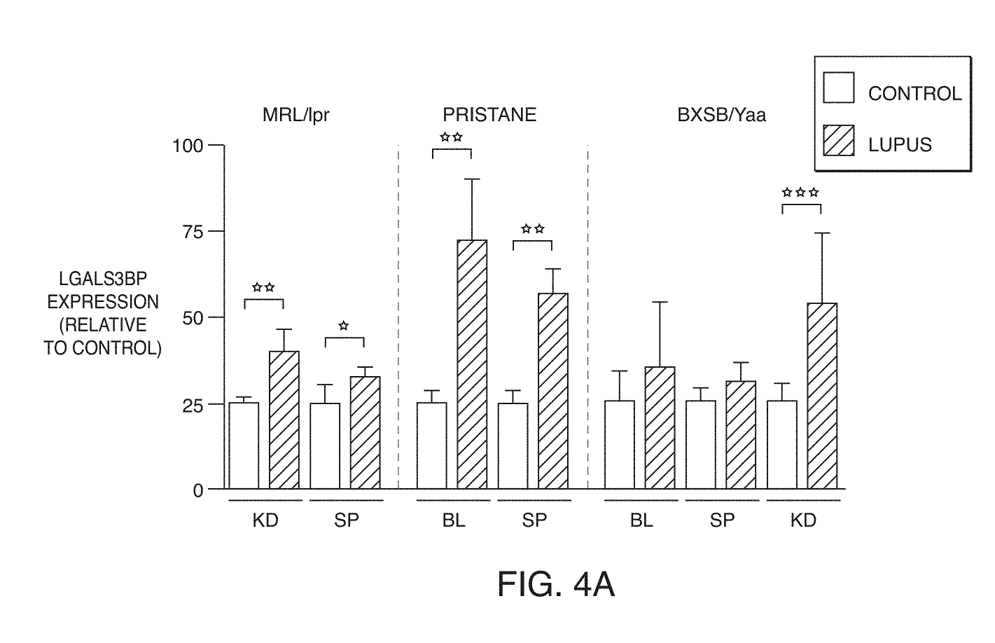

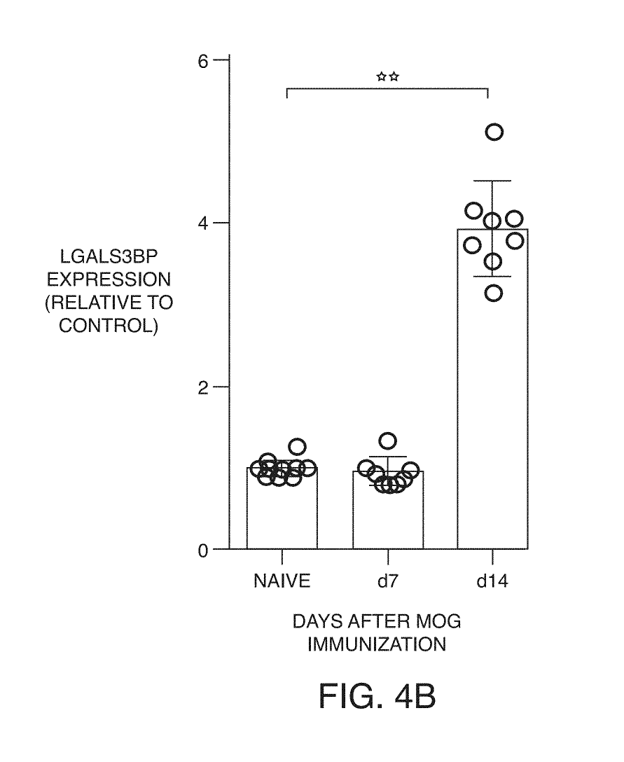

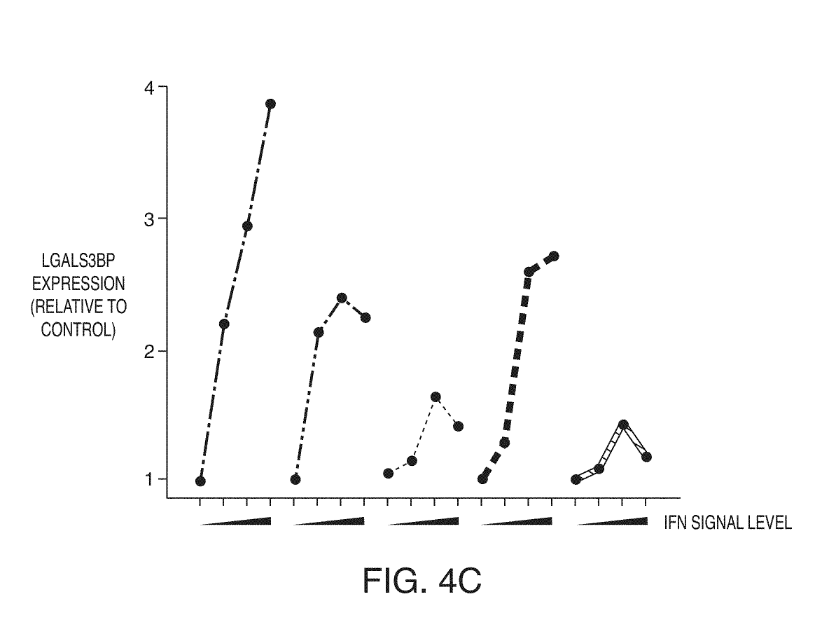

The following experiments tested if the increase of LGALS3BP expression in lupus nephritis patients is conserved in mouse models of SLE. MRL/lpr mice have a mutation in Fas resulting in a defect in lymphocyte apoptosis which ultimately manifests in an SLE-like autoimmune disease. Comparison of MRL/lpr and wildtype C57/BL6 animals showed a significant increase in LGALS3BP expression in kidneys and spleens of diseased animals (FIG. 4A). The same was observed in an induced mouse model of SLE where intraperitoneal injection of pristane leads to autoantibodies, proteinuria and nephritis. These mice also develop an IFN signature detectable in blood and spleen similar to the IFN-induced genes observed in SLE human patients. BXSB/Yaa mice have a duplication of a genetic region that spans the innate RNA sensor TLR7 and develop SLE-like symptoms. TLR7 is known to play an important role in SLE and TLR7 activation leads to the secretion of type I IFNs. Knowing that LGALS3BP expression is inducible by TLR7 stimulation and that its expression correlates with the IFN signature in lupus nephritis human patients LGALS3BP expression was measured across multiple organs in BXSB/Yaa mice. A significant increase in LGALS3BP mRNA was found only in kidney samples of mice that had developed nephritis. Two mice had low nephritis scores and did not show an increase in LGALS3BP expression. In order to evaluate if LGALS3BP expression tracked with IFN-regulated genes, “IFN gene signature scores” were calculated based on the expression of 5 genes (usp18, irf7, ifit1, oas3, bst2). These scores confirmed the same correlation of LGALS3BP expression with IFN scores found in LN patients. Upregulation of IFN-induced genes was also limited to the kidney, further validating the link of LGALS3BP to the IFN response. LGALS3BP was also found to be differentially expressed in multiple sclerosis (MS) human patients and in EAE mice (Raddatz et al., PLUS ONE 2014). This finding was confirmed by immunizing SJL mice with proteolipid protein (PLP) to induce EAE. LGALS3BP expression was significantly increased 14 days after induction of disease (FIG. 4C).

### Example 5: Galectin-3 Inhibition does not Reduce B Cell Viability and Antibody Production

Primary B cells from healthy human donors were stimulated in the presence of galectin-3 inhibitors in order to determine if galectin-3 plays a role in the function of LGALS3BP in B cell biology. Specifically, freshly isolated B cells from healthy volunteers were pre-incubated with galectin-3 (Gal-3) inhibitors for 30 minutes before stimulation with TLR7 agonist for 5 days. Supernatants were harvested and IgG measured by AlphaLISA. Cell viability was measured by CellTiter-Glo (ATP production). None of the inhibitors had an effect on B cell viability or antibody production, indicating that galectin-3 is not directly involved in antibody production by B cells (Table 2).

TABLE 2
Galectin-1 and Galectin-3 Inhibitors do not Induce B cell
Apoptosis and Reduction in Antibody Secretion.

| Compound | Inhibits | IgG production | Viability |
|---|---|---|---|
| LacNAc, N-Acetyl-D-lactosamine | Gal-3 | >10 μM | >10 μM |
| Pectin | Gal-3 | >10 μM | >10 μM |
| (Pienta KJ et al. J Natl Cancer | | | |
| Inst. 1995) | | | |
| Beta n-propal lactoside | Gal-3 | >10 μM | >10 μM |

### Example 6: SP-2, an Anti-LGALS3BP Tumor-Inhibitory Antibody does not Affect B Cell Viability or Antibody Production

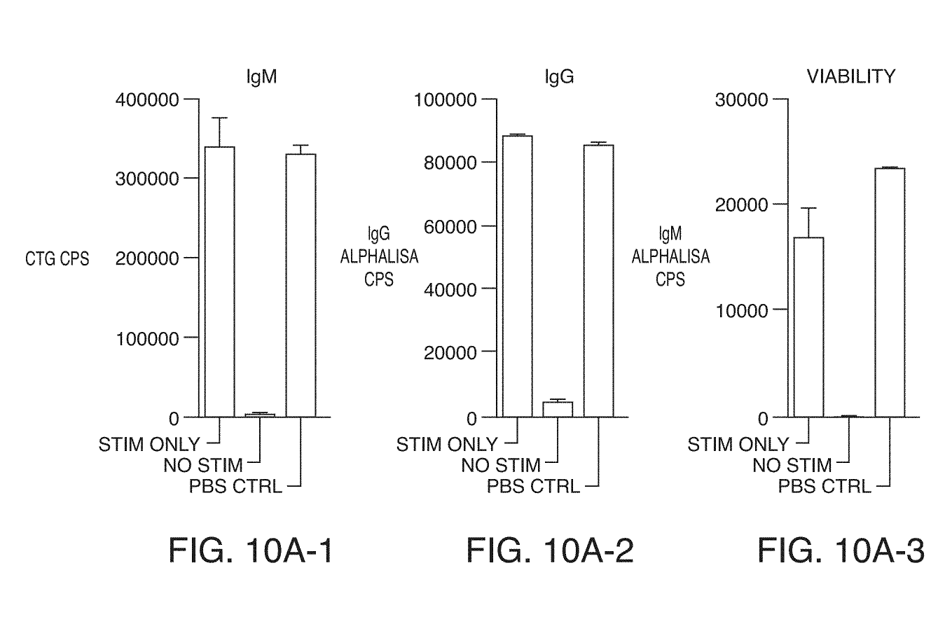

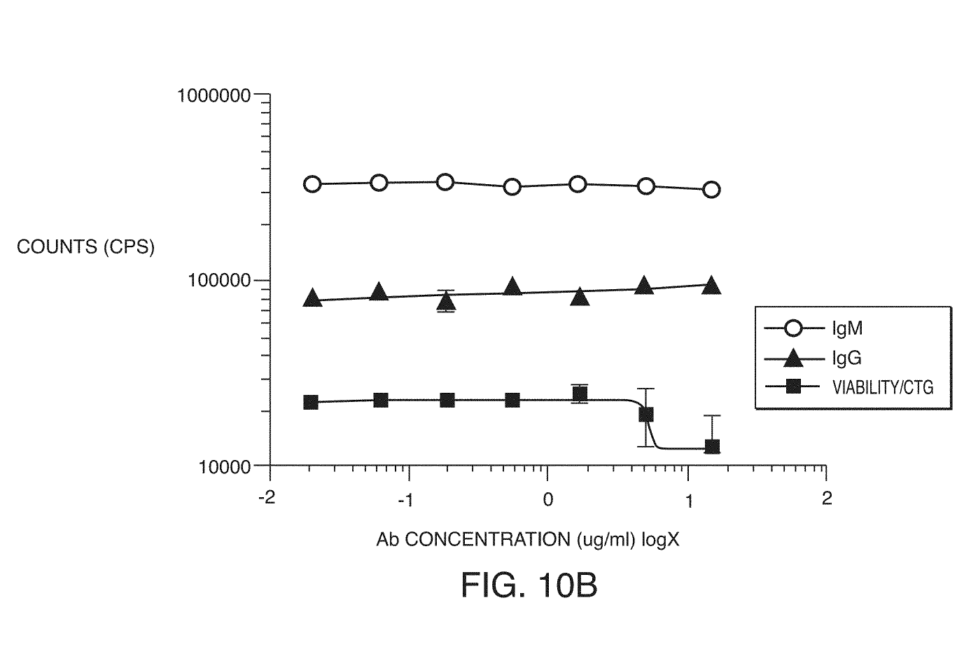

LGALS3BP has been reported to play a role in cancer and SP-2, an anti-LGALS3BP antibody inhibits tumor growth and angiogenesis. SP-2 was tested in a B cell stimulation system and no effect on B cell viability or antibody production was observed (FIG. 10A-1, FIG. 10A-2, FIGS. 10A-3 and 10B-1). Moreover, SP-2 targets the C-terminal domain of LGALS3BP, while the antibody that inhibits B cell viability and antibody production was raised against domain 2, indicating separate functions for different domains of the protein.

For all purposes in the United States of America, each and every publication and patent document cited herein is incorporated by reference for all purposes as if each such publication or document was specifically and individually indicated to be incorporated, herein, by reference.

While the invention has been described with reference to the specific embodiments, changes can be made and equivalents can be substituted to adapt to a particular context or intended use, thereby achieving benefits of the invention without departing from the scope of the claims that follow.

1. A method for modulating LGALS3BP in a subject presenting symptoms of an immune disorder, inflammatory response or autoimmune disease comprising administering an anti-LGALS3BP antibody to said subject under conditions such that at least one symptom of said immune disorder, inflammatory response or disease said is improved.

2. The method of claim 1, wherein, said immune disorder, inflammatory response or autoimmune disease is selected from the group consisting essentially of Graves' disease, myasthenia gravis, vasculitis and Wegener's granulomatosis, neuromyelitis optica, primary sclerosing cholangitis, Sjoegren's syndrome, lupus nephritis and rheumatoid arthritis.

3. A method of treating a patient with SLE, comprising administering to the patient a therapeutically effective amount of an anti-LGALS3BP antibody.

4. The method of claim 3 wherein the amount of anti-LGALS3BP antibody is effective to: (a) inhibit progression of nephritis; (b) stabilize nephritis; or, (c) reverse nephritis, in the patient.

5. The method of claim 3 wherein the amount of anti-LGALS3BP antibody is effective to (a) inhibit progression of proteinuria; (b) stabilize proteinuria; or, (c) reverse proteinuria, in the patient.

6. The method of claim 3 wherein the amount of anti-LGALS3BP antibody is effective to stabilize or decrease, in the patient, a clinical parameter selected from; (a) the patient's blood concentration of urea, creatinine or protein; (b) the patient's urine concentration of protein or blood cells; (c) the patient's urine specific gravity; (d) the amount of the patient's urine; (e) the patient's clearance rate of inulin, creatinine, urea or p-aminohippuric acid; (f) hypertension in the patient; (g) edema in the patient; and, (h) circulating autoantibody levels in the patient.

7. A method of using recombinant LGALS3BP as an adjuvant to enhance the activity of a virally-directed vaccine.
